SEQUENCE LISTING (12 sequences)

SEQ ID NO: 1 - DNA, 20 bp, Fragaria x ananassa
gcgcgcataaggcaacaaag

SEQ ID NO: 2 - DNA, 24 bp, Fragaria x ananassa
gcgaatggcaatgacatcttctct

SEQ ID NO: 3 - DNA, 24 bp, Fragaria x ananassa
gcgtcagccgtagtgatgtagcag

SEQ ID NO: 4 - DNA, 20 bp, Fragaria x ananassa
gcgccagcccctcaaatatc

SEQ ID NO: 5 - DNA, 24 bp, Fragaria x ananassa
gcgaagcataactggcagtatctg

SEQ ID NO: 6 - DNA, 21 bp, Fragaria x ananassa
gcgggcctaggtgatcttgga

SEQ ID NO: 7 - DNA, 23 bp, Fragaria x ananassa
gcgggctgtccacactcctttct

SEQ ID NO: 8 - DNA, 24 bp, Fragaria x ananassa
gcgatgcgtaagtctcttcaaata

SEQ ID NO: 9 - DNA, 24 bp, Fragaria x ananassa
gcgcatcacaatcgccatagaaac

SEQ ID NO: 10 - DNA, 23 bp, Fragaria x ananassa
gcgaacacgccttcaacaaccac

SEQ ID NO: 11 - DNA, 22 bp, Fragaria x ananassa
gcgaaccccattaacagcttca

SEQ ID NO: 12 - DNA, 24 bp, Fragaria x ananassa
gcgatcaaattcccctctaacaat

(19) United States
(12) Plant Patent Application Publication
(10) Pub. No.: US 2013/0025007 P1
(43) Pub. Date: Jan. 24, 2013

(54) Strawberry plant name Rubicon

(50) ;

(71) Applicants: Richard S. Cowles, Mansfield, CT (US); James A. LaMondia, Enfield, CT (US)

(72) Inventors: Richard S. Cowles, Mansfield, CT (US); James A. LaMondia, Enfield, CT (US)

(21) Appl. No.: 13/068,653

(22) Filed: May 17, 2011

(51) Int. Cl. A01H 5/00

(52) U.S. Cl. PLT/208

(57) ABSTRACT

A new and distinct variety of strawberry plant of the short-day type characterized as a mid-season fruiter for Connecticut. Plants are highly vigorous when challenged with the soil-dwelling pathogen complex responsible for black root rot and so are tolerant to this disease. Foliage is not preferred by adult black vine weevils, even in comparison to the non-preferred ‘Delmarvel.’ Flowers are self-fertile with little malformed fruit which is globose conic and usually solid. The fruit has excellent sweetness, acidity, and fragrance. The fruit is moderately soft, making it appropriate for local markets.

### GOVERNMENT INTEREST

This invention was made with government support under the Hatch Project “Managing Insect Pests in Connecticut Suburban Agriculture” awarded by the United States Department of Agriculture (USDA). The government has certain rights in the invention.

### LATIN NAME OF THE GENUS AND SPECIES OF THE PLANT CLAIMED

Fragaria×ananassa

### VARIETY DENOMINATION

‘RUBICON’

### BACKGROUND OF THE INVENTION

This invention relates to a new and distinctive short-day type strawberry (Fragaria×ananassa Duchesne (1766)) cultivar designated as ‘Rubicon’ which is the result of a cross between ‘Primetime’ and ‘Idea’ made in 2000; ‘Idea’ was the female parent.

‘Rubicon’ first fruited at the Connecticut Agricultural Experiment Station, Valley Laboratory, Windsor, Conn., in 2002, where it was designated as ‘IP5’. It was tested continuously under that designation through 2010. Values for growth characteristics are representative of plants grown at the Valley Laboratory, and are likely to vary under different field conditions.

‘Rubicon’ has been propagated asexually by runners and has been tested to a limited extent at a cooperating nursery and with cooperating growers under Test Agreement.

### SUMMARY OF THE INVENTION

‘Rubicon’ plant is a new and distinct variety of strawberry plant of the short-day type characterized as a mid-season fruiter for Connecticut. Plants are highly vigorous when challenged with the soil-dwelling pathogen complex responsible for black root rot and so are tolerant to this disease. Foliage is not preferred by adult black vine weevils, even in comparison to the non-preferred ‘Delmarvel.’ Flowers are self-fertile with little malformed fruit which is globose conic and usually solid. The fruit has excellent sweetness, acidity, and fragrance. The fruit is moderately soft, making it appropriate for local markets.

### BRIEF DESCRIPTION OF THE DRAWINGS

FIG. 1 shows typical growth (A) and flowering (B) characteristics of the plant.

FIG. 2 shows upper (left) and lower (right) surfaces of mature leaves.

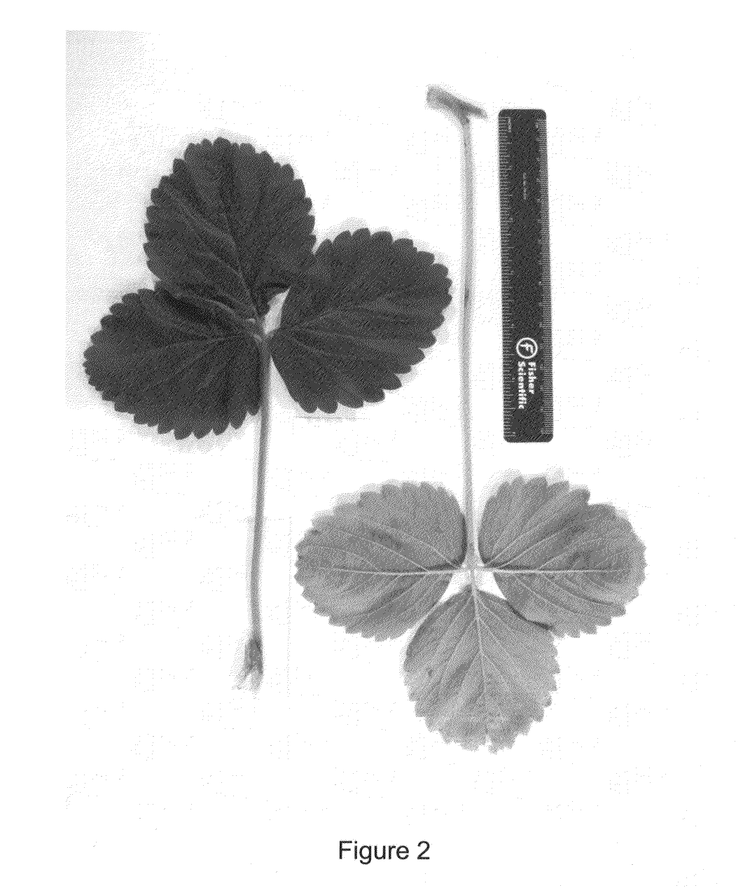

FIG. 3 shows a representative early season fruit cluster (A) and primary fruits in with longitudinal cross-sectional views (B).

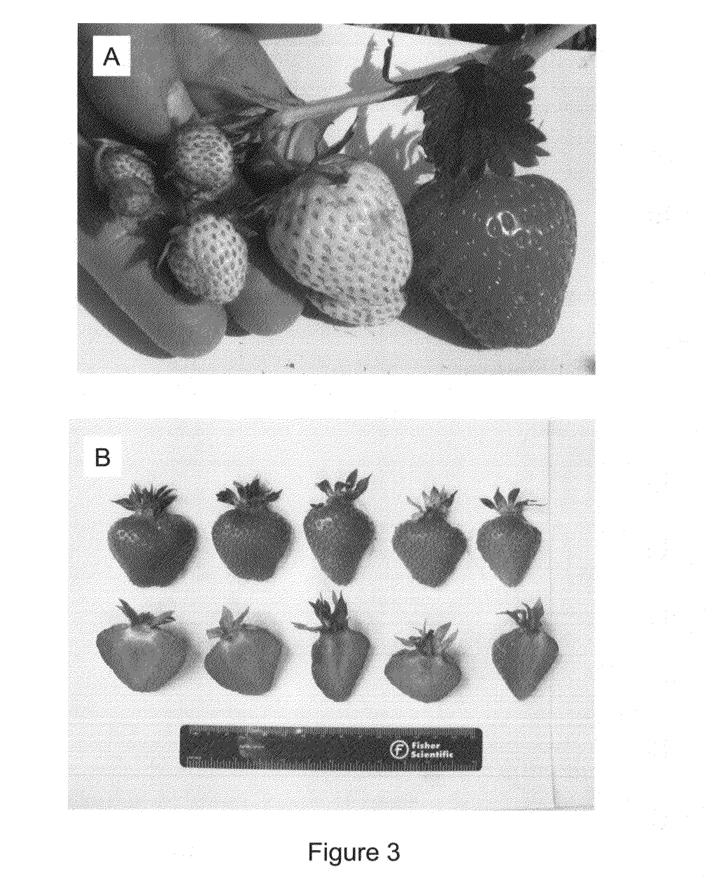

FIG. 4 shows the distinctive DNA banding patterns resulting from fingerprinting with the markers described below and labeled in the figure. Lanes are aligned, from left to right: SS, size standards (measuring base pairs); R, ‘Rubicon’; R, duplicate lane for ‘Rubicon’; P, ‘Primetime’; I, ‘Idea.’

### DETAILED DESCRIPTION OF THE INVENTION

Plants and Foliage

‘Rubicon’ plants are semi-erect in growth habit, with a height of about 25 cm. Terminal leaflets of ‘Rubicon’ are 7.8±1.0 cm long (mean±s.d.), 5.2±0.4 cm wide, and have about 24.4±2.3 serrations. Leaf color on the upper surface is Garden Green (Pantone 19-0230) near veins and a slightly lighter Artichoke Green (Pantone 18-0125) between veins. The color of the leaf underside is Aspen Green (Pantone 17-0215). Runner production in nursery plantings is excellent.

Genetic Typing of Plants

DNA was extracted from young strawberry leaves using a total nucleic acid protocol. Simple sequence repeat markers were used to compare the DNA fingerprint of ‘Rubicon’ strawberry with its parent cultivars, as per the methods of Lewers, et al., 2005. [Lewers, K. S. et al. 2005 Strawberry GenBank-derived and genomic simple sequence repeat (SSR) markers and their utility with strawberry, blackberry, and red and black raspberry. J. Amer. Soc. Hort. Sci 130(1):102-115.] Forward (F) and reverse (R) primers used were:

ARSFL 7:
F: gc gc gcat aa gg caa caa a g
R: gc gaat ggca at ga cat ctt ct ct

ARSFL 10:
F: gc gt ca gcc gt a gt gat gt agca g
R: gc gcc a gcc cct caa at at c

ARSFL 11:
F: gc gaa gc at aact ggca gt at ct g
R: gc gggcct a g gt gat ctt gga

ARSFL 15:
F: gc gggct gt cca cact cct tt ct
R: gc gat gc gt aa gt ct ctt caaat a

ARSFL 17:
F: gc gcat cac aat c gcc at agaaa c
R: gc gaa cac gcctt caaca acc ac

ARSFL 22:
F: gc gaac ccc att aaca gct t ca
R: gc gat caa att cccct ct aacaat

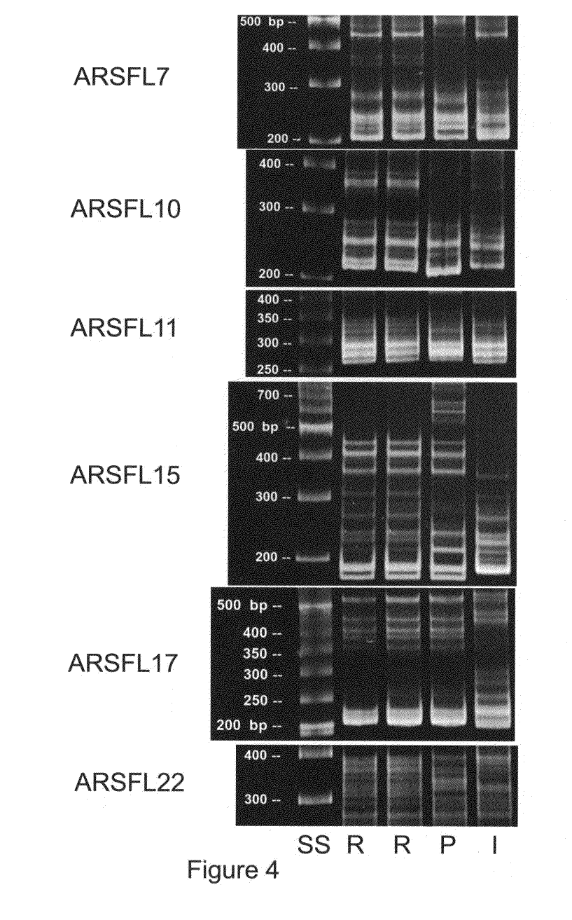

The resulting banding patterns are distinctive and readily separate ‘Rubicon’ from its parents (FIG. 4). In particular, with the ARSFL 7 marker, ‘Rubicon’ has two bands present in the 300-400 bp (base pair) region that are absent for both parents; for the ARSFL 10 marker, ‘Rubicon’ has strong bands present at ˜360 and 370 bp, and a weak band at ˜390 by that are weak in ‘Idea’ and lacking in ‘Primetime’; for ARSFL 11, there are three equally bright bands between 300 and 350 bp, where the middle of these bands is brightest in ‘Idea’ and the smallest is brightest in ‘Primetime’; for ARSFL 15, ‘Rubicon’ is identical to ‘Idea’ in the banding pattern from 200-300 bp, but is identical to ‘Primetime’ in pattern from 350-500 bp; for ARSFL 17, both ‘Rubicon’ and ‘Primetime’ lack bands between 250-300 by that are present in ‘Idea’; for ARSFL22, ‘Rubicon’ is distinct from both parents in the 300-400 by region.

Pest Reaction

‘Rubicon’ plants maintain exceptional vigor when grown in soil containing the pest complex responsible for black root rot and are tolerant of this disease. ‘Rubicon’ leaf disks are less preferred for feeding upon by adult black vine weevils than are leaf disks from the non-preferred variety ‘Delmarvel’ and so these plants are tolerant of black vine weevil. The foliage is less susceptible to leaf scorch than ‘Idea.’

Flowering and Fruiting

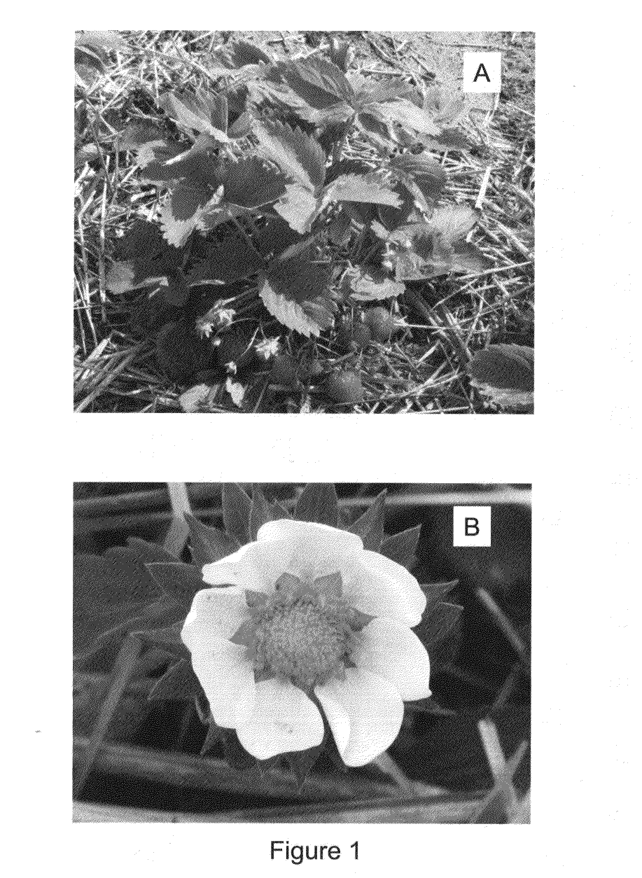

‘Rubicon’ flowers are borne on semi-erect peduncles, 8.2±3.0 cm (mean±s.d.) in length, which are quickly brought down by the weight of the fruit. Primary flowers typically have 10-12 petals (FIG. 1B). Flower measurements for secondary flowers are: corolla diameter, 31.3±2.2 mm; calyx diameter, 37.8±1.9 mm; petal number, 5.8±0.6; petal length, 13.7±0.6 mm; petal width, 11.3±0.6 mm; sepal length, 13.8±1.5 mm; and sepal width, 5.3±1.3 mm. The flowers are self-fertile with ample pollen throughout the season and consequently there are few malformed fruit. Flowers were significantly less susceptible to frost injury than ‘Honeoye’ in 2010 field tests.

Fruit Appearance

‘Rubicon’ fruits are globose conic, with a glossy and attractive skin. Internally, the fruit is usually solid to slightly hollow. The fruit skin color is Aurora Red (Pantone 18-1550) at the darkest, varying to Grenadine (Pantone 17-1558). Internal fruit color varies from Camellia (Pantone 16-1541) to Fiery Red (Pantone 18-1664). The achenes are bright yellow (Aurora, Pantone 12-0642) and are positioned slightly below the fruit surface. The calyx is medium in size borne on a very short neck. The fruit sizes variety as the season progresses, with relatively uniform primary fruit of 24.8±3.7 g, and secondary fruit of 15.8±2.1 g.

Fruit Quality

‘Rubicon’ has flavor comparable to the best flavored varieties. The fruit are characterized by being sweet (brix readings of 9.6±0.42), balanced with substantial tartness, and with an intense aroma. The fruit is moderately soft, with penetrometer readings of 187±9.6 g (using a 3 mm diameter penetrometer probe), but is firmer than the parent ‘Idea’, making it appropriate for local markets.

What is claimed is:

1. A new and distinct variety of strawberry plant named ‘RUBICON,’ as herein described and illustrated and identified by the characteristics set forth above.
